(19) United States
(12) Patent Application Publication
(10) Pub. No.: US 2026/0364318 A1
(43) Pub. Date: Nov. 19, 2026

(54) BALLOON CATHETER

(71) Applicant: KANEKA CORPORATION, Osaka (JP)

(72) Inventors: Chinami Sako, Osaka (JP); Masahiro Kojima, Osaka (JP)

(73) Assignee: KANEKA CORPORATION, Osaka (JP)

(21) Appl. No.: 19/735,040

(22) Filed: Jul. 8, 2026

Related U.S. Application Data
(63) Continuation of application No. PCT/JP2024/039201, filed on Nov. 5, 2024.

(30) Foreign Application Priority Data
Jan. 12, 2024 (JP) 2024-003248

(51) Int. Cl. A61M 25/10; A61M 25/00; A61M 29/02

(52) U.S. Cl. CPC A61M 25/104; A61M 25/0032; A61M 25/1011; A61M 2025/0037; A61M 2025/004; A61M 2025/1061; A61M 2029/025

(57) ABSTRACT

A balloon catheter includes: a balloon portion group including a plurality of balloon portions arranged in parallel; and a proximal shaft portion located on a proximal side with respect to the balloon portion group. The balloon portion group includes an inner balloon portion and an outer balloon portion, the proximal shaft portion includes a guide wire lumen, a first lumen, and a second lumen. In a cross-section perpendicular to a longitudinal direction of the proximal shaft portion, a distance between a centroid of the guide wire lumen and a centroid of the second lumen is longer than a distance between the centroid of the guide wire lumen and a centroid of the first lumen.

### CROSS-REFERENCE TO RELATED APPLICATIONS

This application claims the benefit of priority based on Japanese Patent Application No. 2024-003248, filed on Jan. 12, 2024, and is a continuation of International Patent Application No. PCT/JP2024/039201, filed on Nov. 5, 2024, the entire contents of which are incorporated herein by reference.

### TECHNICAL FIELD

One or more embodiments of the present invention relate to a balloon catheter.

### BACKGROUND

The formation of a stenotic portion, which is hardened by for example calcification, on a vascular wall causes diseases such as angina pectoris and myocardial infarction. One of the treatments for the diseases is angioplasty in which a balloon catheter is used to dilate a stenotic portion. Angioplasty is a widely practiced minimally invasive therapy that does not need a thoracotomy such as bypass surgery.

There is a disease called aortic valve stenosis in which the aortic valve becomes hard due to calcification or the like so that the aortic valve becomes difficult to open and the blood flow is hindered. Examples of the method for treating aortic valve stenosis include an aortic valve replacement surgery involving a thoracotomy and a transcatheter aortic valve replacement surgery in which a biological valve (artificial valve) is indwelled via a catheter and the hardened aortic valve is replaced with the biological valve.

PTL 1 discloses a balloon catheter which is used for dilation of a hardened stenotic portion or indwelling of a biological valve and in which a plurality of outer balloon portions are disposed to surround the outer surface of an inner balloon portion.

### Patent Literature

PTL 1: US Patent Application Publication No. 2012/0209375

However, in the balloon catheter disclosed in PTL 1, as the number of balloons increases, it is difficult to ensure the wall thickness of the portion between lumens of a shaft where an inflation lumen and a guide wire lumen are formed. For this reason, there is fluid leakage from the inflation lumen to the guide wire lumen.

### SUMMARY

One or more embodiments of the present invention have been made in view of the above circumstances, and a balloon catheter that can easily prevent fluid leakage into a guide wire lumen is provided.

A balloon catheter according to one or more aspects of the present invention which can address the above is as follows.
[1] A balloon catheter including:
   a balloon portion group including a plurality of balloon portions arranged in parallel; and
   a proximal shaft portion located on a proximal side with respect to the balloon portion, wherein
   the balloon portion group includes an inner balloon portion including a guide wire tube into which a guide wire is inserted and an outer balloon portion located radially outward of the inner balloon portion,
   the proximal shaft portion includes a guide wire lumen into which the guide wire is inserted, a first lumen that allows a fluid supplied to the inner balloon portion to pass, and a second lumen that allows a fluid supplied to the outer balloon portion to pass, and
   in a cross-section perpendicular to a longitudinal direction of the proximal shaft portion, a distance between a centroid of the guide wire lumen and a centroid of the second lumen is longer than a distance between the centroid of the guide wire lumen and a centroid of the first lumen.

As the distance between the centroid of the guide wire lumen and the centroid of the second lumen is longer than the distance between the centroid of the guide wire lumen and the centroid of the first lumen, it is possible to prevent the portion between the second lumen and the guide wire lumen from becoming too thin because the second lumen is too close to the guide wire lumen. Accordingly, it is possible to easily prevent fluid leakage from the second lumen to the guide wire lumen.

The balloon catheter according to one or more embodiments of the present invention may be any one of the following [2] to [12].
[2] The balloon catheter according to [1], wherein, in a cross-section perpendicular to the longitudinal direction of the proximal shaft portion, the first lumen is present on a line segment connecting the centroid of the guide wire lumen and the centroid of the second lumen.
[3] The balloon catheter according to [1], wherein, in a cross-section perpendicular to the longitudinal direction of the proximal shaft portion, the first lumen is not present on a line segment connecting the centroid of the guide wire lumen and the centroid of the second lumen.
[4] The balloon catheter according to any one of [1] to [3], wherein, in a cross-section perpendicular to the longitudinal direction of the proximal shaft portion, the centroid of the guide wire lumen is offset from a centroid of the proximal shaft portion.
[5] The balloon catheter according to any one of [1] to [4], wherein, in a cross-section perpendicular to the longitudinal direction of the proximal shaft portion, an outer shape of the first lumen is different from an outer shape of the second lumen.
[6] The balloon catheter according to any one of [1] to [5], wherein, in a cross-section perpendicular to the longitudinal direction of the proximal shaft portion, an outer shape of the first lumen is variant.
[7] The balloon catheter according to any one of [1] to [6], wherein, in a cross-section perpendicular to the longitudinal direction of the proximal shaft portion, a cross-sectional area of the first lumen is larger than a cross-sectional area of the second lumen.
[8] The balloon catheter according to any one of [1] to [6], wherein, in a cross-section perpendicular to the longitudinal direction of the proximal shaft portion, a cross-sectional area of the second lumen is larger than a cross-sectional area of the first lumen.
[9] The balloon catheter according to any one of [1] to [8], further including a proximal sleeve portion that connects the balloon portion and the proximal shaft portion, wherein
   the proximal sleeve portion includes a channel that allows a fluid to pass.
[10] The balloon catheter according to any one of [1] to [9], further including
   a distal shaft portion located on a distal side with respect to the balloon portion; and
   a distal sleeve portion that connects the balloon portion and the distal shaft portion.
[11] The balloon catheter according to [10], wherein the distal sleeve portion includes a channel that allows a fluid to pass.
[12] The balloon catheter according to [10], wherein the distal sleeve portion does not include a channel that allows a fluid to pass.

### ADVANTAGEOUS EFFECTS OF INVENTION

The balloon catheter according to one or more embodiments of the present invention can easily prevent fluid leakage from the second lumen to the guide wire lumen.

### BRIEF DESCRIPTION OF DRAWINGS

FIG. 1 is a side view of a balloon catheter according to one or more embodiments of the present invention.

FIG. 2 is an enlarged side view of a balloon portion group included in the balloon catheter illustrated in FIG. 1.

FIG. 3 is a diagram illustrating an end surface of a cut portion taken along the line III-III in FIG. 2.

FIG. 4 is a diagram illustrating an end surface of a cut portion taken along the line IV-IV in FIG. 2.

FIG. 5 is a diagram illustrating an end surface of a cut portion of the balloon catheter illustrated in FIG. 4 according to a modification.

FIG. 6 is a diagram illustrating an end surface of a cut portion of the balloon catheter illustrated in FIG. 4 according to a modification.

FIG. 7 is a side view illustrating a balloon portion group illustrated in FIG. 2 according to a modification.

FIG. 8 is an enlarged cross-sectional view of a balloon portion, a distal sleeve portion, and a proximal sleeve portion included in the balloon catheter illustrated in FIG. 1.

FIG. 9 is a cross-sectional view illustrating a balloon portion, a distal sleeve portion, and a proximal sleeve portion illustrated in FIG. 8 according to a modification.

FIG. 10 is a diagram illustrating an end surface of a cut portion taken along the line X-X illustrated in FIG. 2.

FIG. 11 is a diagram illustrating an end surface of a cut portion of the balloon catheter illustrated in FIG. 10 according to a modification.

FIG. 12 is a diagram illustrating an end surface of a cut portion of the balloon catheter illustrated in FIG. 10 according to a modification.

FIG. 13 is a diagram illustrating an end surface of a cut portion of the balloon catheter illustrated in FIG. 10 according to a modification.

### DETAILED DESCRIPTION

Hereinafter, one or more embodiments of the present invention will be described in detail with reference to the drawings, but the present invention is not limited to the illustrated examples, and various modifications can be made within the suitable range of the gist described above and below and are all included in the technical range of the present invention. In the drawings, hatching, reference numerals, and the like are sometimes omitted for convenience; in such a case, reference is made to the specification and other drawings. Also, the dimensions of various parts in the drawings may differ from the actual dimensions to facilitate understanding of the features of one or more embodiments of the present invention.

A balloon catheter according to one or more aspects of the present invention has a gist in that the balloon catheter includes: a balloon portion group including a plurality of balloon portions arranged in parallel; and a proximal shaft portion located on a proximal side with respect to the balloon portion, in which the balloon portion group includes an inner balloon portion including a guide wire tube into which a guide wire is inserted and an outer balloon portion located radially outward of the inner balloon portion, the proximal shaft portion includes a guide wire lumen into which the guide wire is inserted, a first lumen that allows a fluid supplied to the inner balloon portion to pass, and a second lumen that allows a fluid supplied to the outer balloon portion to pass, and in a cross-section perpendicular to a longitudinal direction of the proximal shaft portion, a distance between a centroid of the guide wire lumen and a centroid of the second lumen is longer than a distance between the centroid of the guide wire lumen and a centroid of the first lumen.

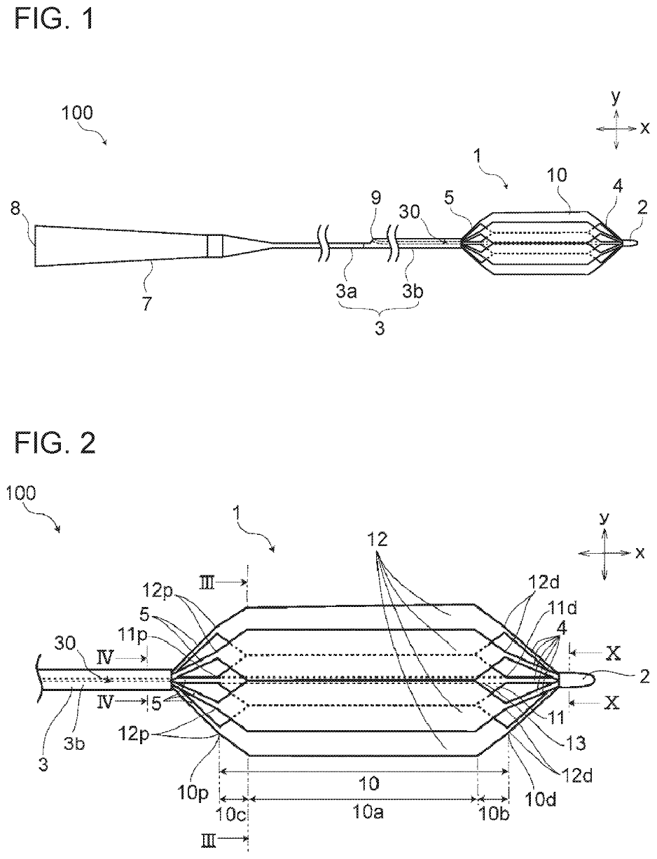

An overall configuration of a balloon catheter according to one or more embodiments of the present invention will be described with reference to FIGS. 1 to 13. In the drawings, a balloon catheter 100 including a balloon portion group 1 and a proximal shaft portion 3 is illustrated. In the drawings, the longitudinal direction of the balloon catheter is indicated by x, the radial direction is indicated by y, and the circumferential direction is indicated by c.

Each of the members and portions included in the balloon catheter 100 also has a longitudinal direction, a radial direction, and a circumferential direction. The longitudinal direction, the radial direction, and the circumferential direction of a member or a portion included in the balloon catheter 100 may coincide with the longitudinal direction x, the radial direction y, and the circumferential direction c of the balloon catheter 100, or may be different from each other. In the present specification, for ease of understanding, a mode is illustrated in which the longitudinal direction, the radial direction, and the circumferential direction of all members and portions other than a distal sleeve portion 4 and a proximal sleeve portion 5 coincide with the longitudinal direction x, the radial direction y, and the circumferential direction c of the balloon catheter 100, respectively.

In the present specification, the proximal side refers to the direction on the user's hand side with respect to the longitudinal direction x of the balloon catheter 100, and the distal side refers to the side opposite to the proximal side, that is, the direction on the treatment target side. In addition, when each member or each part is divided into two equal parts in the longitudinal direction x of the balloon catheter 100, a portion located on the distal side of each member or each part is referred to as a distal portion of each member or each part, and a portion located on the proximal side of each member or each part is referred to as a proximal portion of each member or each part. A distal end of each member or each part is the end of each member or each part that is located on the most distal side. A proximal end of each member or each part is the end of each member or each part that is located on the most proximal side. An end portion includes a peripheral portion of the end. That is, the distal end portion refers to the distal end and the peripheral portion of the distal end, and the proximal end portion refers to the proximal end and the peripheral portion of the proximal end.

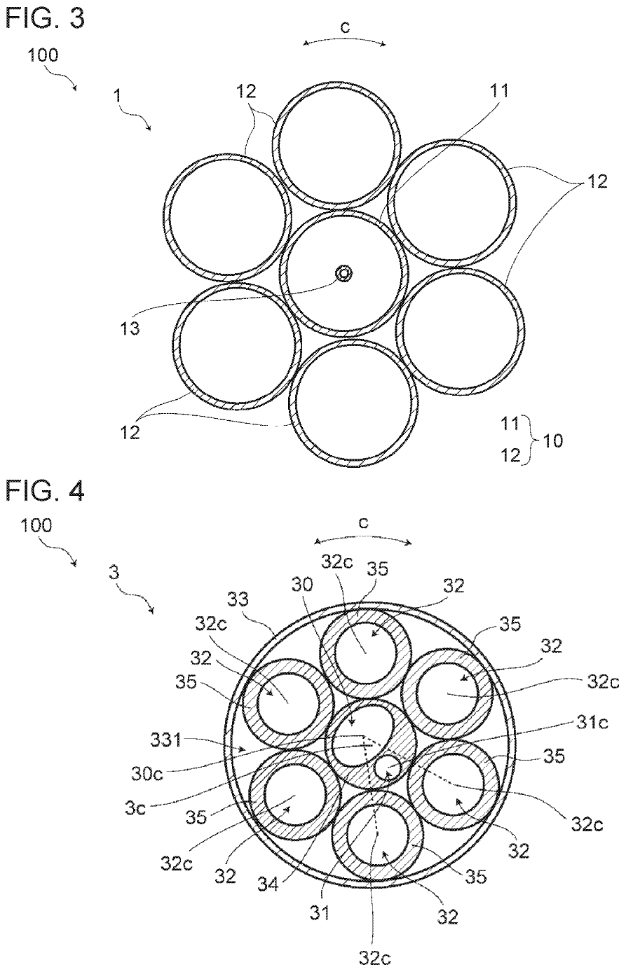

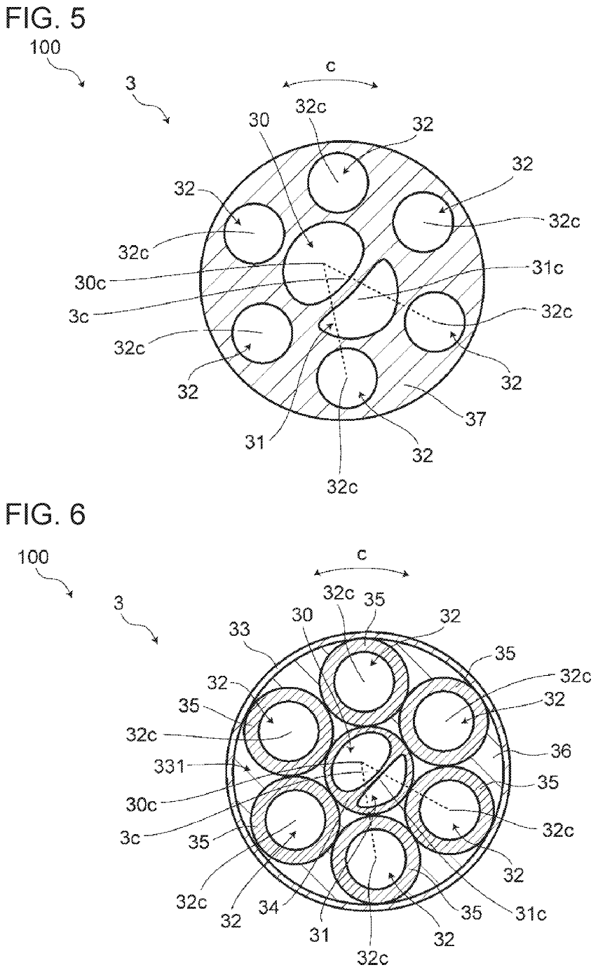

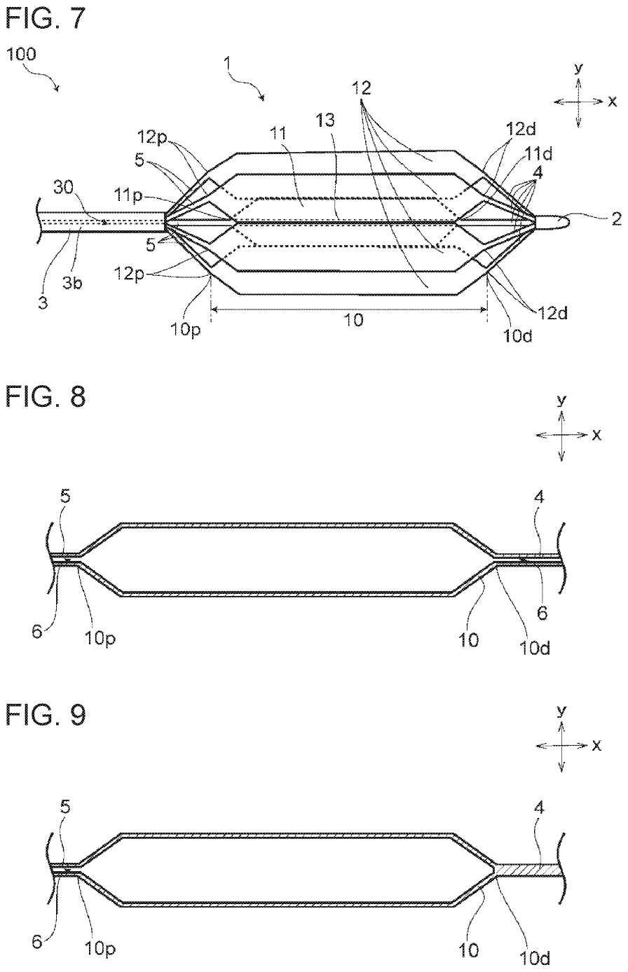

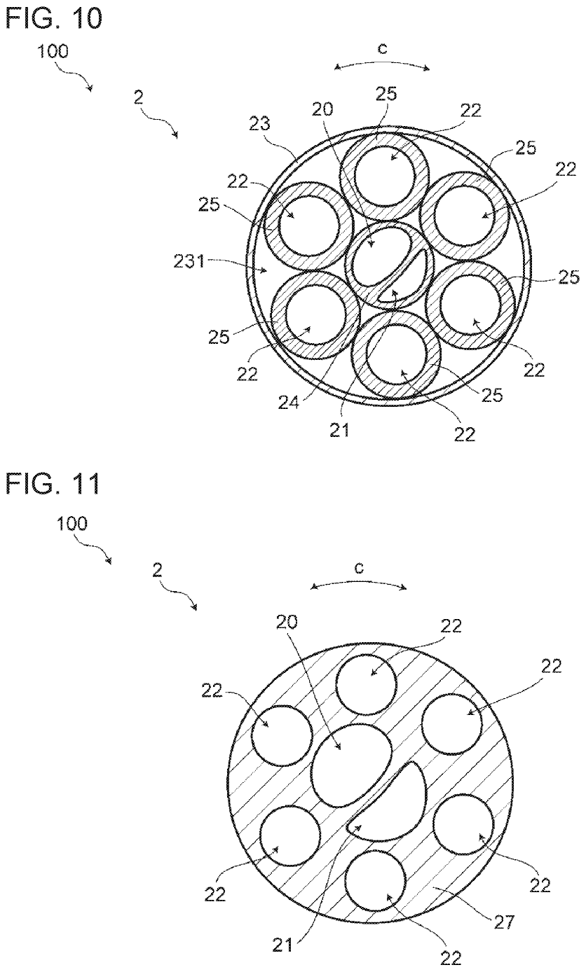

FIG. 1 is a side view of the balloon catheter according to one or more embodiments of the present invention. More specifically, FIG. 1 illustrates a state in which a balloon portion included in the balloon catheter is expanded. FIG. 2 is an enlarged side view of a balloon portion group included in the balloon catheter illustrated in FIG. 1. FIG. 3 is a diagram illustrating an end surface of a cut portion taken along the line III-III in FIG. 2. FIG. 4 is a diagram illustrating an end surface of a cut portion taken along the line IV-IV in FIG. 2. FIGS. 5 and 6 are diagrams illustrating end surfaces of cut portions of the balloon catheter illustrated in FIG. 4 according to modifications. FIG. 7 is a side view illustrating a balloon portion group illustrated in FIG. 2 according to a modification. FIG. 8 is an enlarged cross-sectional view of a balloon portion, a distal sleeve portion, and a proximal sleeve portion included in the balloon catheter illustrated in FIG. 1. FIG. 9 is a cross-sectional view illustrating a balloon portion, a distal sleeve portion, and a proximal sleeve portion illustrated in FIG. 8 according to a modification. More specifically, FIGS. 8 and 9 are cross-sectional views in a cross-section that passes through the central axis of the balloon portion and is parallel to the longitudinal direction of the balloon catheter. FIG. 10 is a diagram illustrating an end surface of a cut portion taken along the line X-X illustrated in FIG. 2. FIGS. 11 to 13 are diagrams illustrating end surfaces of cut portions of the balloon catheter illustrated in FIG. 10 according to modifications.

As illustrated in FIGS. 1 and 2, the balloon catheter 100 includes the balloon portion group 1 and the proximal shaft portion 3.

As illustrated in FIGS. 1 to 3 and 7, the balloon portion group 1 includes a plurality of balloon portions 10 arranged in parallel. The plurality of balloon portions 10 may be arranged in a direction perpendicular to the longitudinal direction x of the balloon catheter 100.

The balloon portion 10 is a portion that expands when a fluid is supplied to the inside thereof and contracts when the fluid is discharged from the inside thereof. FIG. 3 illustrates an end surface of a cut portion of the balloon portion 10 in the direction perpendicular to the longitudinal direction x of the balloon catheter 100. In FIGS. 8 and 9, a cross-section of the balloon portion 10 in a direction parallel to the longitudinal direction x of the balloon catheter 100 is illustrated. In order to control the expansion and contraction of the balloon portion 10, a configuration can be adopted in which a fluid is introduced or discharged using an indeflator (balloon pressure device). As the fluid, for example, a gas such as nitrogen or air, or a liquid such as physiological saline or a mixed solution of a contrast medium and physiological saline can be used. The fluid may be a pressurized fluid that is pressurized by, for example, a pump.

As illustrated in FIG. 2, the balloon portion group 1 includes an inner balloon portion 11 including a guide wire tube 13 into which a guide wire is inserted and an outer balloon portion 12 located radially outward of the inner balloon portion 11. The inner balloon portion 11 refers to, among the plurality of balloon portions 10, the balloon portion 10 in which the guide wire tube 13 into which the guide wire is inserted is provided. The outer balloon portion 12 refers to, among the plurality of balloon portions 10, the balloon portion 10 which is located radially outward of the inner balloon portion 11 and in which the guide wire tube 13 into which the guide wire is inserted is not provided.

As illustrated in FIG. 1, the proximal shaft portion 3 is a portion located on the proximal side with respect to the balloon portion 10.

As illustrated in FIGS. 4 to 6, the proximal shaft portion 3 includes a guide wire lumen 30 into which a guide wire is inserted, a first lumen 31 that allows a fluid supplied to the inner balloon portion 11 to pass, and a second lumen 32 that allows a fluid supplied to the outer balloon portion 12 to pass. In a cross-section perpendicular to the longitudinal direction x of the balloon catheter 100, the guide wire lumen 30, the first lumen 31, and the second lumen 32 may be formed in different regions. The guide wire lumen 30, the first lumen 31, and the second lumen 32 may extend in the longitudinal direction x of the balloon catheter 100.

As illustrated in FIGS. 4 to 6, in a cross-section perpendicular to the longitudinal direction of the proximal shaft portion 3, the distance between a centroid 30c of the guide wire lumen 30 and a centroid 32c of the second lumen 32 is longer than the distance between the centroid 30c of the guide wire lumen 30 and a centroid 31c of the first lumen 31.

As the distance between the centroid 30c of the guide wire lumen 30 and the centroid 32c of the second lumen 32 is longer than the distance between the centroid 30c of the guide wire lumen 30 and the centroid 31c of the first lumen 31, it is possible to prevent the portion formed between the second lumen 32 and the guide wire lumen 30 from becoming too thin because the second lumen 32 is too close to the guide wire lumen 30. Accordingly, it is possible to easily prevent fluid leakage from the second lumen 32 to the guide wire lumen 30.

The balloon catheter 100 according to one or more embodiments of the present invention may be used for dilation of an aortic valve, deformation of a biological valve indwelled in the heart, or destruction of a biological valve. Specifically, the balloon catheter 100 according to one or more embodiments of the present invention may be used for expanding an aortic valve hardened by, for example, calcification, or deforming or destroying, for example, an artificial valve ring of a biological valve to replace the biological valve that has been indwelled in the heart and deteriorated over time.

The balloon catheter 100 has a distal end and a proximal end in the longitudinal direction x.

The number of the balloon portions 10 included in the balloon portion group 1 can be, for example, two or more, three or more, or four or more. The number of the balloon portions 10 included in the balloon portion group 1 can be, for example, 15 or less, 13 or less, or 10 or less. FIGS. 1 to 3 and 7 illustrate a mode in which the seven balloon portions 10 are included in the balloon portion group 1.

As illustrated in FIGS. 2 and 7, the balloon portion 10 has a distal end 10d and a proximal end 10p in the longitudinal direction x of the balloon catheter 100. The distal end of the balloon portion group 1 coincides with the distal end 10d located on the most distal side among the distal ends 10d of the balloon portions 10. The proximal end of the balloon portion group 1 coincides with the proximal end 10p located on the most proximal side among the proximal ends 10p of the balloon portions 10.

Examples of the material forming the balloon portion 10 include polyamide resins such as Nylon 11 and Nylon 12, polyester resins such as polyethylene terephthalate and polybutylene terephthalate, polyurethane resins, thermoplastic elastomers such as polyether block amide copolymers, and combinations thereof.

As illustrated in FIG. 2, the balloon portion 10 may include a straight tube portion 10a, a distal tapered portion 10b that is located on the distal side with respect to the straight tube portion 10a and has an outer diameter decreasing toward the distal side, and a proximal tapered portion 10c that is located on the proximal side with respect to the straight tube portion 10a and has an outer diameter decreasing toward the proximal side. Only some of the balloon portions 10 included in the balloon catheter 100 may include the straight tube portion 10a, the distal tapered portion 10b, and the proximal tapered portion 10c. All of the balloon portions 10 included in the balloon catheter 100 may include the straight tube portion 10a, the distal tapered portion 10b, and the proximal tapered portion 10c.

The straight tube portion 10a may have a hollow cylindrical shape having substantially the same diameter in the longitudinal direction x of the balloon catheter 100, or may have a different diameter in the longitudinal direction x of the balloon catheter 100.

It is preferable that the distal tapered portion 10b and the proximal tapered portion 10c are formed in a hollow conical shape or a hollow truncated conical shape whose diameter decreases away from the straight tube portion 10a.

As illustrated in FIGS. 2 and 7, one inner balloon portion 11 has a distal end 11d and a proximal end 11p.

As illustrated in FIGS. 2 and 7, one outer balloon portion 12 has a distal end 12d and a proximal end 12p.

The number of the inner balloon portions 11 may be, for example, one or more, two or more, or three or more. The number of the inner balloon portions 11 may be, for example, six or less, five or less, or four or less. FIGS. 2, 3, and 7 illustrate a mode in which only one inner balloon portion 11 is provided.

The number of the outer balloon portions 12 can be, for example, one or more, two or more, or three or more. The number of the outer balloon portions 12 may be, for example, 14 or less, 12 or less, or 10 or less. FIGS. 2, 3, and 7 illustrate a mode in which the six outer balloon portions 12 are provided.

When the outer balloon portions 12 are provided, all of the outer balloon portions 12 may be in contact with the inner balloon portion 11. A mode in which some of the outer balloon portions 12 are in contact with the inner balloon portion 11 and the remaining outer balloon portions 12 are not in contact with the inner balloon portion 11 is also allowed. That is, a mode is also allowed in which some of the outer balloon portions 12 are in contact with the inner balloon portion 11 and the remaining outer balloon portions 12 are not in contact with the inner balloon portion 11 and are in contact with the other outer balloon portions 12.

The material forming the inner balloon portion 11 and the material forming the outer balloon portion 12 may be the same or different.

The outer balloon portions 12 may be arranged around the inner balloon portion 11. The inner balloon portion 11 may be present at a position through which the central axis of the balloon portion group 1 passes. The outer balloon portions 12 may be arranged in the circumferential direction c of the balloon catheter 100.

A guide wire is inserted into the guide wire tube 13. The guide wire tube 13 has an inner cavity into which a guide wire is inserted. The inner cavity of the guide wire tube 13 may extend in the longitudinal direction x of the balloon catheter 100.

As a material forming the guide wire tube 13, the material exemplified as the material forming the proximal shaft portion 3 described below can be used.

The guide wire tube 13 may have a shape such as a tubular shape, a hollow cylindrical shape, or a hollow polygonal cylindrical shape.

An X-ray opaque marker may be disposed on the outer surface of the guide wire tube 13 at a position where the inner balloon portion 11 is present in the longitudinal direction x of the balloon catheter 100. The X-ray opaque marker may be disposed on the outer surface of the guide wire tube 13 at the position where the distal end of the straight tube portion of the inner balloon portion 11 is present and at the position where the proximal end of the straight tube portion of the inner balloon portion 11 is present in the longitudinal direction x of the balloon catheter 100. In addition, in the longitudinal direction x of the balloon catheter 100, an X-ray opaque marker may be disposed at the position where the center of the straight tube portion of the inner balloon portion 11 is located.

As illustrated in FIG. 2, the proximal end 11p of the inner balloon portion 11 may be located at the same position as the proximal end 12p of the outer balloon portion 12 in the longitudinal direction x of the balloon catheter 100. As illustrated in FIG. 7, the proximal end 11p of the inner balloon portion 11 may be located on the distal side with respect to the proximal end 12p of the outer balloon portion 12. Although not illustrated, a mode in which the proximal end 11p of the inner balloon portion 11 is located on the proximal side with respect to the proximal end 12p of the outer balloon portion 12 is also allowed.

As illustrated in FIG. 2, the distal end 11d of the inner balloon portion 11 may be located at the same position as the distal end 12d of the outer balloon portion 12 in the longitudinal direction x of the balloon catheter 100. As illustrated in FIG. 7, the distal end 11d of the inner balloon portion 11 may be located on the proximal side with respect to the distal end 12d of the outer balloon portion 12. Although not illustrated, the distal end 11d of the inner balloon portion 11 may be located on the distal side with respect to the distal end 12d of the outer balloon portion 12.

The proximal shaft portion 3 may extend in the longitudinal direction x of the balloon catheter 100.

The proximal shaft portion 3 may have a shape such as a tubular shape, a hollow cylindrical shape, or a hollow polygonal cylindrical shape.

Examples of the proximal shaft portion 3 include: a hollow body formed by arranging one or more wires in a predetermined pattern; one obtained by coating at least one of the inner surface and the outer surface of the hollow body with resin; a resin tube; or a combination thereof, for example, a combination by connecting them in the direction of the longitudinal axis. Examples of the hollow body in which the wires are arranged in a predetermined pattern include a tubular body having a mesh structure in which the wires are crossed or woven, and a coil in which the wires are wound. The wires may be one or more single wires or may be one or more stranded wires. The resin tube can be produced by, for example, extrusion molding. When the proximal shaft portion 3 is a resin tube, the proximal shaft portion 3 can be formed of a single layer or a plurality of layers. A part of the proximal shaft portion 3 in the longitudinal direction x or the circumferential direction c of the balloon catheter 100 may be formed of a single layer, and the other part may be formed of a plurality of layers.

The proximal shaft portion 3 may be made of, for example, a synthetic resin such as a polyolefin resin (e.g., polyethylene or polypropylene), a polyamide resin (e.g., nylon), a polyester resin (e.g., PET), an aromatic polyether ketone resin (e.g., PEEK), a polyether polyamide resin, a polyurethane resin, a polyimide resin, or a fluororesin (e.g., PTFE, PFA, or ETFE), or a metal such as stainless steel, carbon steel, or a nickel-titanium alloy. These materials may be used alone or in combination of two or more kinds thereof.

The proximal shaft portion 3 may be formed of only one tubular member, or may be formed of a plurality of tubular members.

The guide wire lumen 30 may communicate with the inner cavity of the guide wire tube 13 disposed inside the inner balloon portion 11.

The first lumen 31 may communicate with the inside of the inner balloon portion 11. The inner balloon portion 11 may expand due to the fluid supplied through the first lumen 31. The inner balloon portion 11 may contract as the fluid is discharged via the first lumen 31.

The plurality of first lumens 31 may be provided, but only one first lumen 31 may be provided. The number of the first lumens 31 provided may be the same as the number of the inner balloon portions 11.

The second lumen 32 may communicate with the inside of the outer balloon portion 12. The outer balloon portion 12 may expand due to the fluid supplied through the second lumen 32. The outer balloon portion 12 may contract as the fluid is discharged through the second lumen 32.

Only one second lumen 32 may be provided, but the plurality of second lumens 32 may be provided. The number of the second lumens 32 provided may be the same as the number of the outer balloon portions 12. In a cross-section perpendicular to the longitudinal direction of the proximal shaft portion 3, the distance between the centroid 30c of the guide wire lumen 30 and the centroid 32c of the second lumen 32 is longer than the distance between the centroid 30c of the guide wire lumen 30 and the centroid 31c of the first lumen 31, so that the second lumens 32 can be easily arranged concentrically and at equal intervals.

In a cross-section perpendicular to the longitudinal direction of the proximal shaft portion 3, the second lumen 32 may be located outward of the first lumen 31 in the radial direction of the proximal shaft portion 3.

The outer shape of the guide wire lumen 30, the outer shape of the first lumen 31, and the outer shape of the second lumen 32 in a cross-section perpendicular to the longitudinal direction of the proximal shaft portion 3 may be a circular shape, a polygonal shape including a triangular shape and a square shape, a semicircular shape, a fan shape, a wedge shape, a convex shape, a crescent shape, or a combination thereof.

As illustrated in FIGS. 5 and 6, the outer shape of the first lumen 31 in a cross-section perpendicular to the longitudinal direction of the proximal shaft portion 3 may be different from the outer shape of the second lumen 32. In a cross-section perpendicular to the longitudinal direction of the proximal shaft portion 3, the outer shape of the first lumen 31 may be different from the outer shape of the guide wire lumen 30. In a cross-section perpendicular to the longitudinal direction of the proximal shaft portion 3, the outer shape of the second lumen 32 may be different from the outer shape of the guide wire lumen 30. In a cross-section perpendicular to the longitudinal direction of the proximal shaft portion 3, the outer shape of the first lumen 31, the outer shape of the second lumen 32, and the outer shape of the guide wire lumen 30 may be different. With such a configuration, the proximal shaft portion 3 can be easily reduced in diameter.

As illustrated in FIGS. 5 and 6, in a cross-section perpendicular to the longitudinal direction of the proximal shaft portion 3, the outer shape of the first lumen 31 may be variant. Variant means a shape other than a perfect circle, and shapes such as an ellipse or an oval are not included in the perfect circle. Although the term “circular shape” or the like may be used in this specification, the circular shape is defined to include shapes such as a perfect circular shape, an elliptical shape, and an oval shape.

As illustrated in FIG. 5, in a cross-section perpendicular to the longitudinal direction of the proximal shaft portion 3, the cross-sectional area of the first lumen 31 may be larger than the cross-sectional area of the second lumen 32. Accordingly, the inner balloon portion 11 can be more easily expanded at an earlier timing than the outer balloon portion 12.

As illustrated in FIG. 4, in a cross-section perpendicular to the longitudinal direction of the proximal shaft portion 3, the cross-sectional area of the second lumen 32 may be larger than the cross-sectional area of the first lumen 31. Accordingly, the outer balloon portion 12 can be easily expanded at an earlier timing than the inner balloon portion 11.

Although not illustrated, in a cross-section perpendicular to the longitudinal direction of the proximal shaft portion 3, the cross-sectional area of the first lumen 31 and the cross-sectional area of the second lumen 32 may be the same. Accordingly, the inner balloon portion 11 and the outer balloon portion 12 can be easily expanded at the same time.

As illustrated in FIGS. 2, 4, and 6, the proximal shaft portion 3 may include an outer tube 33 having an inner cavity 331 extending in the longitudinal direction x of the balloon catheter 100, a first tube 34 disposed in the inner cavity 331 of the outer tube 33, and a second tube 35 disposed in the inner cavity 331 of the outer tube 33. The second tube 35 may be located radially outward of the first tube 34. In a cross-section perpendicular to the longitudinal direction of the proximal shaft portion 3, the second tube 35 may be located outward of the first tube 34 in the radial direction of the proximal shaft portion 3.

The material forming the outer tube 33, the material forming the first tube 34, and the material forming the second tube 35 may be the same or different.

The guide wire lumen 30 and the first lumen 31 may be formed in the first tube 34, and the second lumen 32 is formed in the second tube 35.

The plurality of first tubes 34 may be provided, but only one first tube 34 may be provided. The number of the first tubes 34 provided may be the same as the number of the inner balloon portions 11. The distal end portion of the first tube 34 may be connected to the proximal end portion of the proximal sleeve portion 5 connected to the inner balloon portion 11. Accordingly, a fluid can be supplied to the inside of the inner balloon portion 11 via the first lumen 31 of the first tube 34 and a channel 6 of the proximal sleeve portion 5 described below.

Only one second tube 35 may be provided, but the plurality of second tubes 35 may be provided. The number of the second tubes 35 provided may be the same as the number of the outer balloon portions 12. The distal end portion of the second tube 35 may be connected to the proximal end portion of the proximal sleeve portion 5 connected to the outer balloon portion 12. Accordingly, the fluid can be supplied to the inside of the outer balloon portion 12 via the second lumen 32 of the second tube 35 and the channel 6 of the proximal sleeve portion 5.

As illustrated in FIG. 6, a filling material 36 may be disposed in the space surrounded by the inner surface of the outer tube 33, the outer surface of the first tube 34, and the outer surface of the second tube 35.

As the filling material 36, those exemplified as the material forming the balloon portion 10 and the material forming the proximal shaft portion 3 can be used.

As illustrated in FIG. 4, a gap may be present in the space surrounded by the inner surface of the outer tube 33, the outer surface of the first tube 34, and the outer surface of the second tube 35. The inner surface of the outer tube 33, the outer surface of the first tube 34, and the outer surface of the second tube 35 may be exposed to the gap.

As illustrated in FIG. 5, the proximal shaft portion 3 may include a tubular member 37 including the guide wire lumen 30 into which the guide wire is inserted, the first lumen 31 that allows the fluid supplied to the inner balloon portion 11 to pass, and the second lumen 32 that allows the fluid supplied to the outer balloon portion 12 to pass. In this case, the proximal end portion of the proximal sleeve portion 5 connected to the inner balloon portion 11 and the distal end portion of the proximal shaft portion 3 may be connected to each other such that the channel 6 of the proximal sleeve portion 5 connected to the inner balloon portion 11 communicates with the first lumen 31. In addition, the proximal end portion of the proximal sleeve portion 5 connected to the outer balloon portion 12 and the distal end portion of the proximal shaft portion 3 may be connected to each other such that the channel 6 of the proximal sleeve portion 5 connected to the outer balloon portion 12 communicates with the second lumen 32. With the above configuration, the fluid can be supplied to the inside of the inner balloon portion 11 via the first lumen 31 and the channel 6 of the proximal sleeve portion 5. In addition, the fluid can be supplied to the inside of the outer balloon portion 12 via the second lumen 32 and the channel 6 of the proximal sleeve portion 5.

As illustrated in FIGS. 5 and 6, in a cross-section perpendicular to the longitudinal direction of the proximal shaft portion 3, the first lumen 31 may be present on a line segment connecting the centroid 30c of the guide wire lumen 30 and the centroid 32c of the second lumen 32. In FIGS. 5 and 6, the line segment connecting the centroid 30c of the guide wire lumen 30 and the centroid 32c of the second lumen 32 is indicated by a broken line. Accordingly, the cross-sectional area of the first lumen 31 in a cross-section perpendicular to the longitudinal direction of the proximal shaft portion 3 can be relatively large so that the inner balloon portion 11 can be easily expanded at an earlier timing than the outer balloon portion 12.

As illustrated in FIG. 4, in a cross-section perpendicular to the longitudinal direction of the proximal shaft portion 3, the first lumen 31 does not need to be present on the line segment connecting the centroid 30c of the guide wire lumen 30 and the centroid 32c of the second lumen 32. More specifically, in a cross-section perpendicular to the longitudinal direction of the proximal shaft portion 3, the first lumen 31 may be present between two line segments that connect the centroid 30c of the guide wire lumen 30 and the centroid 32c of the second lumen 32 and are adjacent to each other in the circumferential direction c of the balloon catheter 100. In FIG. 4, the line segment connecting the centroid 30c of the guide wire lumen 30 and the centroid 32c of the second lumen 32 is indicated by a broken line. Accordingly, the cross-sectional area of the first lumen 31 in a cross-section perpendicular to the longitudinal direction of the proximal shaft portion 3 can be relatively small so that the outer balloon portion 12 can be easily expanded at an earlier timing than the inner balloon portion 11.

As illustrated in FIGS. 4 to 6, in a cross-section perpendicular to the longitudinal direction of the proximal shaft portion 3, the centroid 30c of the guide wire lumen 30 may be offset from a centroid 3c of the proximal shaft portion 3. Although not illustrated, there is an acceptable mode in which the centroid 30c of the guide wire lumen 30 coincides with the centroid 3c of the proximal shaft portion 3 in a cross-section perpendicular to the longitudinal direction of the proximal shaft portion 3. In a cross-section perpendicular to the longitudinal direction of the proximal shaft portion 3, the guide wire lumen 30 may be formed in a region including the centroid 3c of the proximal shaft portion 3.

As illustrated in FIGS. 1, 2, and 7, the balloon catheter 100 may include the proximal sleeve portion 5 connecting the balloon portion 10 and the proximal shaft portion 3.

The plurality of proximal sleeve portions 5 may be connected to one balloon portion 10, but it is preferable that only one proximal sleeve portion 5 is connected to one balloon portion 10.

It is preferable that the plurality of proximal sleeve portions 5 are connected to one proximal shaft portion 3.

It is preferable that one proximal sleeve portion 5 is connected to one balloon portion 10, and it is more preferable that the distal end portion of one proximal sleeve portion 5 is connected to the proximal end portion of one balloon portion 10. It is preferable that one proximal shaft portion 3 is connected to the plurality of proximal sleeve portions 5, and it is more preferable that the distal end portion of one proximal shaft portion 3 is connected to the proximal end portions of the plurality of proximal sleeve portions 5.

As illustrated in FIGS. 8 and 9, the proximal sleeve portion 5 may include the channel 6 that allows the fluid to pass. Although not illustrated in detail, the channel 6 may communicate from the distal end to the proximal end of the proximal sleeve portion 5. Accordingly, the fluid can be supplied to the inside of the balloon portion 10 via the channel 6 of the proximal sleeve portion 5.

The proximal sleeve portion 5 can have a shape such as a tubular shape, a hollow cylindrical shape, or a hollow polygonal cylindrical shape.

As the material forming the proximal sleeve portion 5, those exemplified as the material forming the balloon portion 10 and the material forming the proximal shaft portion 3 can be used.

As illustrated in FIGS. 1, 2, and 7, the balloon catheter 100 may include a distal shaft portion 2 located on the distal side with respect to the balloon portion 10.

As illustrated in FIG. 1, the distal shaft portion 2 is a portion located on the distal side with respect to the balloon portion 10.

The distal shaft portion 2 may extend in the longitudinal direction x of the balloon catheter 100. The distal shaft portion 2 may or may not include a lumen that allows the fluid to pass after the fluid is supplied to the balloon portion 10.

The distal shaft portion 2 can have a shape such as a tubular shape, a hollow cylindrical shape, a hollow polygonal cylindrical shape, a conical shape, a truncated conical shape, or a tapered shape.

As illustrated in FIG. 1, in the longitudinal direction x of the balloon catheter 100, the length of the distal shaft portion 2 may be shorter than the length of the proximal shaft portion 3.

As a material forming the distal shaft portion 2, the material exemplified as the material forming the proximal shaft portion 3 can be used.

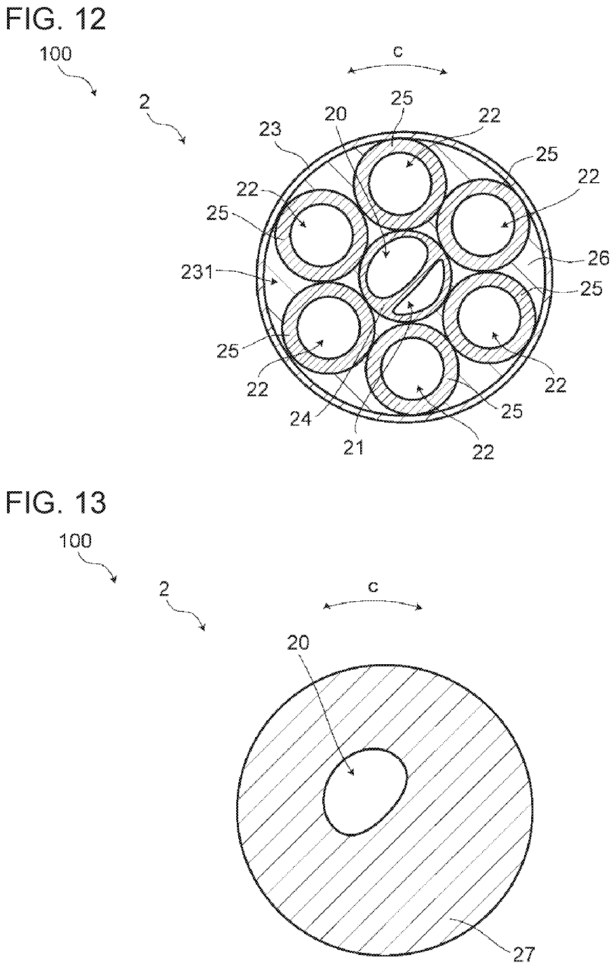

As illustrated in FIGS. 10 to 13, the distal shaft portion 2 can be configured to include a guide wire lumen 20 into which a guide wire is inserted. As illustrated in FIGS. 10 to 12, the distal shaft portion 2 may include the guide wire lumen 20 into which the guide wire is inserted, a first lumen 21 that allows the fluid to pass after the fluid is supplied to the inner balloon portion 11, and a second lumen 22 that allows the fluid to pass after the fluid is supplied to the outer balloon portion 12. In this case, in a cross-section perpendicular to the longitudinal direction x of the balloon catheter 100, the guide wire lumen 20, the first lumen 21, and the second lumen 22 may be formed in different regions. As illustrated in FIG. 13, from the viewpoint of ease of reducing the diameter, it is preferable that the distal shaft portion 2 includes only the guide wire lumen 20 into which the guide wire is inserted. The guide wire lumen 20, the first lumen 21, and the second lumen 22 may extend in the longitudinal direction x of the balloon catheter 100.

The guide wire lumen 20 may communicate with the inner cavity of the guide wire tube 13 disposed inside the inner balloon portion 11.

The first lumen 21 may communicate with the inside of the inner balloon portion 11.

The plurality of first lumens 21 may be provided, or only one first lumen 21 may be provided. The number of the first lumens 21 provided may be the same as the number of the inner balloon portions 11.

The second lumen 22 may communicate with the inside of the outer balloon portion 12.

Only one second lumen 22 may be provided, or the plurality of second lumens 22 may be provided. The number of the second lumens 22 provided may be the same as the number of the outer balloon portions 12.

In a cross-section of the distal shaft portion 2 taken in a direction perpendicular to the longitudinal direction of the proximal shaft portion 3, the second lumen 22 may be located outward of the first lumen 21 in the radial direction of the proximal shaft portion 3.

The outer shape of the guide wire lumen 20, the outer shape of the first lumen 21, and the outer shape of the second lumen 22 in a cross-section of the distal shaft portion 2 taken in a direction perpendicular to the longitudinal direction of the proximal shaft portion 3 may be a circular shape, a polygonal shape including a triangular shape and a square shape, a semicircular shape, a fan shape, a wedge shape, a convex shape, a crescent shape, or a combination thereof.

As illustrated in FIGS. 2, 10, and 12, the distal shaft portion 2 may include an outer tube 23 including an inner cavity 231 extending in the longitudinal direction x of the balloon catheter 100, a first tube 24 disposed in the inner cavity 231 of the outer tube 23, and a second tube 25 disposed in the inner cavity 231 of the outer tube 23. In a cross-section of the distal shaft portion 2 taken in a direction perpendicular to the longitudinal direction of the proximal shaft portion 3, the second tube 25 may be located outward of the first tube 24 in the radial direction of the proximal shaft portion 3.

The material forming the outer tube 23, the material forming the first tube 24, and the material forming the second tube 25 may be the same or different.

The guide wire lumen 20 and the first lumen 21 may be formed in the first tube 24, and the second lumen 22 may be formed in the second tube 25.

The plurality of first tubes 24 may be provided, or only one first tube 24 may be provided. The number of the first tubes 24 provided may be the same as the number of the inner balloon portions 11. The proximal end portion of the first tube 24 may be connected to the distal end portion of the distal sleeve portion 4 connected to the inner balloon portion 11. Accordingly, the fluid is supplied from the inner balloon portion 11 to the first lumen 21 of the first tube 24 via the channel 6 of the distal sleeve portion 4 described below.

Only one second tube 25 may be provided, or the plurality of second tubes 25 may be provided. The number of the second tubes 25 provided may be the same as the number of the outer balloon portions 12. The proximal end portion of the second tube 25 may be connected to the distal end portion of the distal sleeve portion 4 connected to the outer balloon portion 12. Accordingly, the fluid is supplied from the outer balloon portion 12 to the second lumen 22 of the second tube 25 via the channel 6 of the distal sleeve portion 4.

As illustrated in FIG. 12, a filling material 26 may be disposed in the space surrounded by the inner surface of the outer tube 23, the outer surface of the first tube 24, and the outer surface of the second tube 25.

As the filling material 26, those exemplified as the material forming the balloon portion 10 and the material forming the proximal shaft portion 3 can be used.

As illustrated in FIG. 10, a gap may be present in the space surrounded by the inner surface of the outer tube 23, the outer surface of the first tube 24, and the outer surface of the second tube 25. The inner surface of the outer tube 23, the outer surface of the first tube 24, and the outer surface of the second tube 25 may be exposed to the gap.

As illustrated in FIG. 11, the distal shaft portion 2 may include a tubular member 27 including the guide wire lumen 20 into which the guide wire is inserted, the first lumen 21 that allows the fluid to pass after the fluid is supplied to the inner balloon portion 11, and the second lumen 22 that allows the fluid to pass after the fluid is supplied to the outer balloon portion 12. In this case, the distal end portion of the distal sleeve portion 4 connected to the inner balloon portion 11 and the proximal end portion of the distal shaft portion 2 may be connected to each other such that the channel 6 of the distal sleeve portion 4 connected to the inner balloon portion 11 communicates with the first lumen 21. Further, the distal end portion of the distal sleeve portion 4 connected to the outer balloon portion 12 and the proximal end portion of the distal shaft portion 2 may be connected to each other such that the channel 6 of the distal sleeve portion 4 connected to the outer balloon portion 12 communicates with the second lumen 22. With the above configuration, the fluid after being supplied to the inner balloon portion 11 is supplied to the first lumen 21 via the channel 6 of the distal sleeve portion 4. In addition, the fluid after being supplied to the outer balloon portion 12 is supplied to the second lumen 22 via the channel 6 of the distal sleeve portion 4.

The guide wire lumen 20 may penetrate in the longitudinal direction x of the balloon catheter 100.

The first lumen 21 and the second lumen 22 may have open proximal ends and closed distal ends. That is, it is preferable that no fluid is supplied to the distal side with respect to the distal shaft portion 2. As a result, the fluid is likely to be accumulated inside the balloon portion 10, and the balloon portion 10 is likely to be expanded.

As illustrated in FIGS. 1, 2, and 7, the balloon catheter 100 may include the distal sleeve portion 4 that connects the balloon portion 10 and the distal shaft portion 2.

The plurality of distal sleeve portions 4 may be connected to one balloon portion 10, but it is preferable that only one distal sleeve portion 4 is connected to one balloon portion 10.

It is preferable that the plurality of distal sleeve portions 4 are connected to one distal shaft portion 2.

It is preferable that one distal sleeve portion 4 is connected to one balloon portion 10, and it is more preferable that the proximal end portion of one distal sleeve portion 4 is connected to the distal end portion of one balloon portion 10. It is preferable that one distal shaft portion 2 is connected to the plurality of distal sleeve portions 4, and it is more preferable that the proximal end portion of one distal shaft portion 2 is connected to the distal end portions of the plurality of distal sleeve portions 4.

As illustrated in FIG. 8, the distal sleeve portion 4 may include the channel 6 that allows the fluid to pass. Although not illustrated in detail, the channel 6 may communicate from the distal end to the proximal end of the distal sleeve portion 4. The channel 6 may be present only in a part between the distal end and the proximal end of the distal sleeve portion 4.

As illustrated in FIG. 9, the distal sleeve portion 4 does not need to include the channel 6 that allows the fluid to pass. This makes it easier to reduce the diameter of the distal sleeve portion 4.

The distal sleeve portion 4 can have a shape such as a cylindrical shape, a polygonal cylindrical shape, a tubular shape, a hollow cylindrical shape, or a hollow polygonal cylindrical shape.

As the material forming the distal sleeve portion 4, those exemplified as the material forming the balloon portion 10 and the material forming the proximal shaft portion 3 can be used.

As illustrated in FIG. 1, a hub 7 may be connected to the proximal portion of the proximal shaft portion 3. Although not illustrated, the hub 7 may have a bifurcated structure. In addition, the hub 7 may include a fluid injection portion 8 that allows the fluid supplied to the inside of the balloon portion 10 to be injected. The fluid injection portion 8 may be connected to the above-described indeflator.

The proximal shaft portion 3 and the hub 7 may be fixed to each other. For example, the proximal shaft portion 3 and the hub 7 can be fixed to each other by, for example, adhesion with an adhesive, welding, or a screw. In particular, the proximal shaft portion 3 and the hub 7 may be fixed to each other by adhesion. The proximal shaft portion 3 and the hub 7 are fixed to each other by adhesion, and therefore, in a case where the material forming the proximal shaft portion 3 is different from the material forming the hub 7, for example, when the proximal shaft portion 3 is made of a highly flexible material and the hub 7 is made of a highly rigid material, the strength of the connection between the proximal shaft portion 3 and the hub 7 can be increased so that it is easy to enhance the durability of the balloon catheter 100.

FIG. 1 illustrates a mode in which a guide wire port 9 is formed in the middle between the distal end and the proximal end of the proximal shaft portion 3. The guide wire port 9 communicates with the guide wire lumen 30 of the proximal shaft portion 3. FIG. 1 illustrates the balloon catheter 100 of what is called a rapid exchange type. The proximal shaft portion 3 may include a first proximal shaft portion 3a and a second proximal shaft portion 3b located on the distal side with respect to the first proximal shaft portion 3a. The first proximal shaft portion 3a and the second proximal shaft portion 3b may be separate members, and a distal end portion of the member forming the first proximal shaft portion 3a may be connected to a proximal end portion of the member forming the second proximal shaft portion 3b. Alternatively, the first proximal shaft portion 3a and the second proximal shaft portion 3b may be formed by a single member.

As illustrated in FIG. 2, the proximal end portion of the guide wire tube 13 may be connected to the distal end portion of the proximal shaft portion 3. The proximal end portion of the guide wire tube 13 and the distal end portion of the proximal shaft portion 3 may be directly connected to each other without another member interposed therebetween, or may be indirectly connected to each other with another member interposed therebetween. When the proximal shaft portion 3 includes the first proximal shaft portion 3a and the second proximal shaft portion 3b, the proximal end portion of the guide wire tube 13 may be connected to the distal end portion of the second proximal shaft portion 3b.

As illustrated in FIG. 2, the distal end portion of the guide wire tube 13 may be connected to the proximal end portion of the distal shaft portion 2. The distal end portion of the guide wire tube 13 and the proximal end portion of the distal shaft portion 2 may be connected directly without another member interposed therebetween, or may be connected indirectly with another member interposed therebetween.

Although not illustrated, the balloon catheter 100 may be of what is called an over-the-wire type in which the guide wire lumen 30 is formed from the distal end to the proximal end of the proximal shaft portion 3. When the balloon catheter 100 is of the over-the-wire type, the guide wire lumen 30 of the proximal shaft portion 3 may extend to the position where the hub 7 is present in the longitudinal direction x of the balloon catheter 100. The first lumen 31 and the second lumen 32 of the proximal shaft portion 3 may extend to the middle from the distal end to the proximal end of the proximal shaft portion 3. The proximal end of the first lumen 31 and the proximal end of the second lumen 32 of the proximal shaft portion 3 may be located on the distal side with respect to the distal end of the hub 7. The first lumen 31 and the second lumen 32 of the proximal shaft portion 3 may extend to a position where the hub 7 is present in the longitudinal direction x of the balloon catheter 100.

Coating may be applied to the outer surface of the proximal shaft portion 3 and the outer surface of the distal shaft portion 2. As illustrated in FIG. 1, when the balloon catheter 100 is of a rapid exchange type, coating may be applied to at least one of the outer surfaces of the first proximal shaft portion 3a and the second proximal shaft portion 3b, or coating may be applied to both the outer surfaces of the first proximal shaft portion 3a and the second proximal shaft portion 3b. When the balloon catheter 100 is of an over-the-wire type, coating may be applied to the whole or only a part of the outer surface of the proximal shaft portion 3. Coating may be applied to the whole or only a part of the outer surface of the distal shaft portion 2.

The coating applied to the outer surface of the proximal shaft portion 3 and the outer surface of the distal shaft portion 2 may be hydrophilic coating or hydrophobic coating depending on the purpose. The coating can be applied by immersing the proximal shaft portion 3 and the distal shaft portion 2 in a hydrophilic coating agent or a hydrophobic coating agent, by applying a hydrophilic coating agent or a hydrophobic coating agent to the outer surface of the proximal shaft portion 3 and the outer surface of the distal shaft portion 2, or by coating the outer surface of the proximal shaft portion 3 and the outer surface of the distal shaft portion 2 with a hydrophilic coating agent or a hydrophobic coating agent. A drug or an additive may be added to a coating agent.

Examples of a hydrophilic coating agent include hydrophilic polymers such as polyvinyl alcohol, polyethylene glycol, polyacrylamide, polyvinylpyrrolidone, and methyl vinyl ether-maleic anhydride copolymer, and a hydrophilic coating agent obtained by a combination thereof.

Examples of a hydrophobic coating agent include polytetrafluoroethylene (PTFE), fluorinated ethylene propylene (FEP), perfluoroalkoxyalkane (PFA), silicone oil, hydrophobic urethane resin, carbon coating, diamond coating, diamond-like carbon (DLC) coating, ceramic coating, and a substance having a low surface free energy and terminated with an alkyl group or a perfluoroalkyl group.

Although the disclosure has been described with respect to only a limited number of embodiments, those skilled in the art, having benefit of this disclosure, will appreciate that various other embodiments may be devised without departing from the scope of the present disclosure. Accordingly, the scope of the invention should be limited only by the attached claims.

### REFERENCE SIGNS LIST

1 balloon portion group
10 balloon portion
10a straight tube portion
10b distal tapered portion
10c proximal tapered portion
10d distal end
10p proximal end
11 inner balloon portion
11d distal end
11p proximal end
12 outer balloon portion
12d distal end
12p proximal end
13 guide wire tube
2 distal shaft portion
20 guide wire lumen
21 first lumen
22 second lumen
23 outer tube
231 inner cavity
24 first tube
25 second tube
26 filling material
27 tubular member
3 proximal shaft portion
3a first proximal shaft portion
3b second proximal shaft portion
3c centroid of proximal shaft portion
30 guide wire lumen
30c centroid of guide wire lumen
31 first lumen
31c centroid of first lumen
32 second lumen
32c centroid of second lumen
33 outer tube
331 inner cavity
34 first tube
35 second tube
36 filling material
37 tubular member
4 distal sleeve portion
5 proximal sleeve portion
6 channel
7 hub
8 fluid injection portion
9 guide wire port
100 balloon catheter
x longitudinal direction of balloon catheter
y radial direction of balloon catheter
c circumferential direction of balloon catheter

1. A balloon catheter comprising:
a balloon portion group including a plurality of balloon portions arranged in parallel; and
a proximal shaft portion located on a proximal side with respect to the balloon portion group, wherein
the balloon portion group includes an inner balloon portion including a guide wire tube configured to receive a guide wire and an outer balloon portion located radially outward of the inner balloon portion,
the proximal shaft portion includes a guide wire lumen configured to receive the guide wire, a first lumen that allows a fluid supplied to the inner balloon portion to pass, and a second lumen that allows a fluid supplied to the outer balloon portion to pass, and
in a cross-section perpendicular to a longitudinal direction of the proximal shaft portion, a distance between a centroid of the guide wire lumen and a centroid of the second lumen is longer than a distance between the centroid of the guide wire lumen and a centroid of the first lumen.

2. The balloon catheter according to claim 1, wherein, in a cross-section perpendicular to the longitudinal direction of the proximal shaft portion, the first lumen is present on a line segment connecting the centroid of the guide wire lumen and the centroid of the second lumen.

3. The balloon catheter according to claim 1, wherein, in a cross-section perpendicular to the longitudinal direction of the proximal shaft portion, the first lumen is not present on a line segment connecting the centroid of the guide wire lumen and the centroid of the second lumen.

4. The balloon catheter according to claim 1, wherein, in a cross-section perpendicular to the longitudinal direction of the proximal shaft portion, the centroid of the guide wire lumen is offset from a centroid of the proximal shaft portion.

5. The balloon catheter according to claim 1, wherein, in a cross-section perpendicular to the longitudinal direction of the proximal shaft portion, an outer shape of the first lumen is different from an outer shape of the second lumen.

6. The balloon catheter according to claim 1, wherein, in a cross-section perpendicular to the longitudinal direction of the proximal shaft portion, an outer shape of the first lumen is variant.

7. The balloon catheter according to claim 1, wherein, in a cross-section perpendicular to the longitudinal direction of the proximal shaft portion, a cross-sectional area of the first lumen is larger than a cross-sectional area of the second lumen.

8. The balloon catheter according to claim 1, wherein, in a cross-section perpendicular to the longitudinal direction of the proximal shaft portion, a cross-sectional area of the second lumen is larger than a cross-sectional area of the first lumen.

9. The balloon catheter according to claim 1, further comprising a proximal sleeve portion that connects the balloon portion group and the proximal shaft portion, wherein
the proximal sleeve portion includes a channel that allows a fluid to pass.

10. The balloon catheter according to claim 1, further comprising:
a distal shaft portion located on a distal side with respect to the balloon portion group; and
a distal sleeve portion that connects the balloon portion group and the distal shaft portion.

11. The balloon catheter according to claim 10, wherein the distal sleeve portion includes a channel that allows a fluid to pass.

12. The balloon catheter according to claim 10, wherein the distal sleeve portion does not include a channel that allows a fluid to pass.
